(19) United States
(12) Patent Application Publication
(10) Pub. No.: US 2023/0320975 A1
(43) Pub. Date: Oct. 12, 2023

(54) SPRAYABLE COMPOSITION OF A THROAT SPRAY

(71) Applicant: PM-INTERNATIONAL AG, Schengen (LU)

(72) Inventors: Rolf Sorg, Schengen (LU); Tobias Kühne, Morbach (DE); Rudi Wajda, Mannheim (DE); Horst Meinhardt, Herschbach (DE)

(21) Appl. No.: 18/022,925

(22) PCT Filed: May 11, 2021
(86) PCT No.: PCT/EP2021/062442

§ 371 Date: Feb. 23, 2023

(30) Foreign Application Priority Data
Aug. 27, 2020 (DE) 10 2020 122 469.9
Aug. 27, 2020 (DE) 20 2020 104 968.2

(51) Int. Cl. A61K 8/9789; A61Q 11/00

(52) U.S. Cl. CPC A61K 8/9789; A61Q 11/00; A61K 2800/92

(57) ABSTRACT

A composition consisting of a polar phase and a non-polar phase, wherein the polar phase contains at least 40% by weight of glycerol and at least 20% by weight of water, based on the total weight of the polar phase, the composition contains from 0.5 g to 4 g of lecithin per 100 g of composition, and the non-polar phase contains at least one extract selected from an extract of mint (Mentha arvensis or Mentha x piperita), an extract of clove tree (Syzygium aromaticum), an extract of ginger (Zingiber officinale) and an extract of thyme (Thymus vulgaris).

The present application is a national stage application filed under 35 U.S.C. § 371 of International Application No. PCT/EP2021/062442, filed May 11, 2021, which claims priority to German Patent Application No. 10 2020 122 469.9, filed Aug. 27, 2020 and to German Patent Application No. 20 2020 104 968.2, filed Aug. 27, 2020. Each of the aforementioned Patent Applications is incorporated by reference in its entirety for all purposes.

### DESCRIPTION

The disclosure relates to a composition, a spray containing the composition according to the disclosure, the use of a spray device for dispensing a composition according to the disclosure, and a non-medical method for conditioning a portion of the human body.

Liquid compositions for use in the oral cavity and throat are known. These compositions are marketed, inter alia, as sprays under the synonymous names “throat spray” or “oral spray”. These compositions or sprays may be either food products, dietary supplements or medical products.

For example, various sprays exist that contain liquid compositions containing disinfectant agents, such as cetylpyridinium, dequalinium, hexamidine, and/or local anesthetics, such as lidocaine, and/or nonsteroidal anti-inflammatory agents, such as flurbiprofen. Some compositions in the sprays contain vegetable oils, such as chamomile oil, or other fat-soluble compounds, such as cinnamaldehyde or levomenthol. Some compositions in the sprays contain vitamins and/or amino acids and/or minerals.

In addition to the sprays already mentioned for use in the oral cavity and throat, there are also skin sprays containing similar compositions with active ingredients such as hydrocortisone.

The known compositions generally have an aqueous base. The use of these compositions therefore requires that the compositions are microbiologically stable, i.e. that the growth of bacterial or fungal cultures in the compositions is prevented. In order to ensure the microbiological stability of the compositions even after opening, the addition of preservatives is usually required.

Artificial preservatives, such as sorbic acid and derivatives thereof, parabens (derivatives of 4-hydroxybenzoic acid, especially 4-hydroxybenzoic acid methyl ester and 4-hydroxybenzoic acid propyl ester), sodium edetate and glycerol triacetate, are usually added for preservation. Some of these compounds are controversial because of possible intolerances, among other reasons. Moreover, since preservatives are usually lipophilic, they pass at least partially into the lipophilic phase in compositions with aqueous and lipophilic phases. To ensure that the concentration of preservative in the aqueous phase is sufficiently high to effect preservation, two-phase systems are therefore usually formulated with a high preservative content.

The known compositions usually contain water-soluble and fat-soluble compounds. Therefore, the addition of solubilizers is also usually required, such as ethanol, propylene glycol and derivatives thereof, macrogol and derivatives thereof, cellulose and derivatives thereof (e.g. hypromellose), cyclodextrin and derivatives thereof, anionic, cationic and nonionic surfactants. However, surfactants can affect the effectiveness of preservatives. A product containing ethanol is also not available to everyone; pregnant women, children and people with addiction histories are excluded. Other solubilizers are also at least controversial, especially when taken orally, e.g. propylene glycol and sodium dodecyl sulfate because of skin irritation, macrogol glycerol ricinoleate because of stomach upset, diarrhea, and skin and mucous membrane irritation. There is also the problem that biphasic compositions, i.e. mixtures that have an aqueous phase and a lipid phase, are not physically stable over time and phase separation occurs some time after manufacture.

Some compounds are used because they have both preservative and solubilizing properties, e.g., tetraalkylammonium compounds, such as benzalkonium, benzethonium, cetylalkonium, cetylpyridinium, cetyltrimethylammonium, dequalinium, and the alcohols ethanol and propylene glycol.

Artificial sweeteners, monosaccharides or disaccharides are also often added to the compositions to improve the taste.

Concentrated aqueous solutions of the disaccharide sucrose are also used as a basis for sprays because the sucrose in high concentration is suitable for preservation and as a flavor carrier. The disadvantage, of course, is that such a composition is cariogenic and also causes blood sugar spikes, which is particularly disadvantageous for diabetics. Diabetics, however, are particularly exposed to some risks, so that especially for diabetics, as far as possible, any product that has a nutritional, health or disease-preventing benefit should be suitable.

One obstacle to the use of liquid compositions in sprays is that the compositions must be sprayable via a spray device. Compositions with high viscosity, e.g. oils, or compositions containing solids are often not sprayable or can at most be applied in the form of a strong jet and without atomization.

Against this background, one object of the disclosure is to provide a composition suitable for spraying a portion of the human body, in particular the oral cavity and pharynx, and having advantages over compositions of known throat sprays. The composition should be microbiologically stable and both water-soluble and fat-soluble compounds should be included or soluble or at least dispersible in the composition.

Efforts to solve this object result in a composition characterized in that the composition consists of a polar phase and a non-polar phase, wherein the polar phase contains at least 40% by weight of glycerol and at least 20% by weight of water, based on the total weight of the polar phase, the composition contains 0.5 g to 4 g lecithin per 100 g of the composition, and the non-polar phase or the composition contains at least one extract selected from an extract of mint (Mentha arvensis or Mentha x piperita), an extract of clove tree (Syzygium aromaticum), an extract of ginger (Zingiber officinale) and an extract of thyme (Thymus vulgaris).

The liquid composition consists of a polar phase containing water and a non-polar phase containing non-polar compounds, and yet-at least when observed by eye-forms a homogeneous mixture. In other words, the composition contains water-soluble and fat-soluble compounds, with the compounds being dissolved or at least dispersed. The composition is therefore physically stable. The special base of glycerol, water and lecithin in certain proportions contributes to this. The composition is also microbiologically stable. The glycerol content and the at least one extract in particular contribute to the microbiological stability. The at least one extract may contain essential oil. Thus, only by means of naturally occurring ingredients, a microbiologically and physically stable composition suitable for oral application is obtained. As a result, the composition is particularly well accepted physiologically and psychologically and is particularly well tolerated. The composition has a low risk of intolerances such as allergies. Both water-soluble and fat-soluble compounds dissolve in the composition and can be included without the need for additional solubilizers or surfactants. Thus, the basis of the composition allows hydrophilic and hydrophobic compounds to be incorporated into the composition to further modify the properties of the composition or to use the composition as a delivery system for certain compounds. Synthetic or non-naturally occurring additives for preservation or solubilization are not required. The composition exhibits good taste and odor even in the absence of monosaccharides and disaccharides, and thus is non-cariogenic and does not cause blood sugar spikes. Above all, the composition is sprayable. Despite the high content of relatively viscous glycerol and the oily extract(s), the composition can be applied in the form of a spray. The special base also allows for a long interaction with a sprayed body part and an exchange between the composition and the sprayed body part. The at least one extract provides a variety of positive effects in addition to preservation. The ingredients of the extracts can act at the application site, improve taste, breath odor (halitosis) and mouthfeel, and may even have antibacterial and/or antiviral effects.

The extract from the mint may be an extract from the field mint (Mentha arvensis) or from the peppermint (Mentha x piperita). In the context of the disclosure, an extract from the field mint (Mentha arvensis) is preferred.

The extract from field mint (Mentha arvensis) is preferably an extract from the aerial parts, preferably the flowering aerial parts. This may be mint oil according to the European Pharmacopoeia. The extract may be obtained, for example, by steam extraction. The ingredients of the extract may have antioxidant, possibly also antibacterial and/or antiviral, effects and impart a fragrant taste and breath.

In another embodiment of the disclosure, an extract from peppermint (Mentha x piperita) is used instead of the extract from field mint (Mentha arvensis). The extract from peppermint is preferably an extract from the aerial parts, preferably the flowering aerial parts. This may be peppermint oil according to the European Pharmacopoeia. The extract may be obtained, for example, by steam extraction. The ingredients of the extract may have antioxidant and possibly antibacterial and/or antiviral activities and impart a fragrant taste and breath.

The extract from the clove tree (Syzygium aromaticum) is preferably an extract from the cloves, preferably clove oil. Preferably, the extract is obtained from the flower buds, more preferably the dried flower buds. This may be cloves according to the European Pharmacopoeia. The ingredients of the extract may have antioxidant effects and impart a fragrant taste and breath.

The extract from ginger (Zingiber officinale) is preferably an extract from ginger rhizomes, preferably from dried ginger rhizomes. This may be ginger rhizome according to the European Pharmacopoeia. Preferably, the extract is a CO₂ distraction extract. The ingredients of the extract may have antioxidant effects.

The extract from thyme (Thymus vulgaris) is preferably an extract from the flowering aerial parts. This may be thyme oil according to the European Pharmacopoeia. The extract may be obtained, for example, by steam extraction. The ingredients of the extract may possibly have antibacterial and antiviral effects.

Lecithin is a preparation containing phosphatidylcholines as the main ingredient. Phosphatidylcholines are 1,2-diacyl-sn-glycero-3-phosphocholine, phospholipids in which phosphoric acid is esterified with diacylglycerol and choline. Lecithin is an additive approved for foodstuffs in general (except certain foodstuffs) under the number E 322 of the German Additives Regulation (Zusatzstoff-Zulassungsverordnung-ZZulV 1998).

The composition according to the disclosure contains 0.5 g to 4 g, more preferably 1 g to 3.5 g, even more preferably 1.5 g to 3.0 g or 2.0 g to 3.0 g of lecithin per 100 g of the composition. The lecithin is predominantly present in the boundary region between the polar and non-polar phases. In other words, in addition to the polar phase and the non-polar phase, the composition also comprises a lecithin phase consisting of the lecithin in the composition. The lecithin has a phosphatidylcholine content of from 30% to 100% by weight. In other words, the composition preferably contains 0.2 g to 4 g, more preferably 0.3 g to 3.5 g, even more preferably 0.5 g to 3.0 g or 0.6 g to 3.0 g of phosphatidylcholine per 100 g of the composition. In one embodiment, the composition according to the disclosure contains 1 g to 3 g, preferably 1 g to 2 g, of phosphatidylcholine per 100 g of the composition. Preferably, deoiled lecithin fractions are used as lecithin. This means that the lecithin is preferably essentially free of non-polar lipids such as fatty acids and triglycerides. The content of non-polar lipids such as fatty acids and triglycerides is preferably less than 3% by weight. The lecithin contains a content of polar lipids (acetone-insoluble substance) of 90 to 100% by weight. The lecithin may be derived from a source selected from soy, sunflower, canola, fish, milk and/or eggs, with non-animal sources such as soy, sunflower and canola being particularly preferred. In addition to the main ingredient phosphatidylcholine, lecithin may contain phosphatidylethanolamine, phosphatidylinositol, phosphatidylserine, sterols, fats and oils, depending on the source. For example, a lecithin from soy may contain 40-50% by weight phosphatidylcholine, about 10% by weight phosphatidylethanolamine, about 5% by weight phosphatidylinositol, about 1-2% by weight phosphatidylserine, and further sterols, fats and oils. In contrast, lecithin obtained from egg yolks consists essentially of phosphatidylcholine.

Water and glycerol, optionally also polysaccharides and water-soluble vitamins, are components of the polar phase.

The non-polar phase can consist of fatty acids and their esters, in particular triglycerides (triacylglycerols) and ethyl esters, fat-soluble vitamins as well as carotenoids, e.g. lutein and zeaxanthin, vitamin A (retinol) and its derivatives retinal, retinyl palmitate and retinyl acetate, vitamin E as a collective term for tocopherols and tocotrienols (including alpha-, beta-, gamma-, delta-tocopherols and tocotrienols, both as natural mixtures and synthetically in pure form), vitamin E derivatives such as alpha-, beta-, gamma- and delta-tocopherol acetate (both in optically pure form and as racemates), vitamin D2 (ergocalciferol), vitamin D3 (cholecalciferol), vitamin K1 (phylloquinone), vitamin K2 (menaquinone), vitamin K3 (menadione), coenzyme Q 10 H (ubiquinol), ubiquinone-10 and perfluorocarbons. In the present disclosure, suitable sources of the components of the non-polar phase are in particular the extract or extracts from mint (Mentha arvensis or Mentha x piperita), in particular mint oil and/or peppermint oil, from the clove tree (Syzygium aromaticum), in particular clove oil, from ginger (Zingiber officinale) and from thyme (Thymus vulgaris), in particular thyme oil, as well as coconut oil and fat-soluble vitamins.

The composition or spray is a dietary supplement or food.

The composition according to the disclosure is liquid or a liquid at room temperature (25° C.).

Embodiments within this specification may be combined with each other in any manner, unless the subject matter and description of the embodiments clearly indicate otherwise.

The verbs “contain” and “comprise” and their conjugations also contain the verb “consist of” with its conjugations.

Preferred embodiments are also reproduced in the claims.

The polar phase of the composition according to the disclosure contains at least 40% by weight of glycerol, based on the total weight of the polar phase. A high content of glycerol supports microbiological preservation of the composition. The inventors have discovered that by using the extract(s), the amount of preservative glycerol in the polar phase and the composition can be decreased and the amount of water can be increased. This makes the composition sprayable. Sprayable in the sense of the disclosure means that the composition can be delivered in the form of droplets or an atomizing spray, e.g., from a spray device commonly used for throat sprays. In other words, the composition can be atomized using a spray device (atomizer) commonly used for throat sprays. Not sprayable in the sense of the disclosure are compositions that are not delivered or are delivered in the form of a strong jet by usual spray devices for throat sprays.

Preferably, the composition is not only sprayable and suitable to be taken orally by humans, but also suitable to be sprayed into the human throat.

Preferably, the non-polar phase (and composition) contains an extract of mint (Mentha arvensis or Mentha x piperita), an extract of clove tree (Syzygium aromaticum), an extract of ginger (Zingiber officinale) and an extract of thyme (Thymus vulgaris). The combination of these extracts makes particularly good use of the effects described above.

More preferably, the non-polar phase (and composition) contains an extract of field mint (Mentha arvensis), an extract of clove tree (Syzygium aromaticum), an extract of ginger (Zingiber officinale) and an extract of thyme (Thymus vulgaris). The combination of these extracts makes particularly good use of the effects described above.

Preferably, the non-polar phase (and composition) contains from 0.4 g to 10.0 g, preferably from 1.5 g to 7.0 g, of the extract or extracts in total per 100 g of the composition.

Preferably, the proportion of the non-polar phase in the composition is at most 25% by weight, more preferably at most 20% by weight, still more preferably at most 15% by weight, wherein the polar phase and the lecithin form the difference ad 100% by weight. In a preferred embodiment, the proportion of the non-polar phase in the composition relative to the total weight of the composition is from 0.4% to 10.0% by weight, preferably from 1.5% to 7.0% by weight, wherein the polar phase and the lecithin make up the difference ad 100% by weight. In a more preferred embodiment, the proportion of the non-polar phase in the composition is from 0.9% by weight to 15% by weight, preferably from 2.0% by weight to 12.0% by weight, wherein the polar phase and the lecithin make up the difference ad 100% by weight.

Preferably, the polar phase contains from 40% to 78% by weight, more preferably from 45% to 75% by weight, even more preferably from 50% to 70% by weight, especially preferably from 53% to 67% by weight, most preferably from 56% to 64% by weight, of glycerol, based on the total weight of the polar phase.

Preferably, the composition (and the polar phase) contains from 0.01 g to 2.0 g of polysaccharides per 100 g of the composition. The inventors have found that polysaccharides in this amount have a positive effect on the stability of the composition and the sprayability. Preferably, the polysaccharides are β-glucans, more preferably (1,3)-(1,6)-beta-D-glucans. β-Glucans are polysaccharides built up from 3-D-glucose units (β-D-glucopyranose).

Preferably, the non-polar phase (and composition) contains from 0.1 g to 4.0 g, preferably from 1.0 g to 2.5 g, of an extract of field mint (Mentha arvensis) per 100 g of the composition.

Preferably, the non-polar phase (and composition) contains from 0.1 g to 2.0 g, preferably from 0.2 g to 1.2 g, of an extract from the clove tree (Syzygium aromaticum) per 100 g of the composition.

Preferably, the non-polar phase (and composition) contains from 0.1 g to 2.0 g, preferably from 0.2 g to 0.8 g, of an extract of ginger (Zingiber officinale) per 100 g of the composition.

Preferably, the non-polar phase (and the composition) contains from 0.1 g to 2.0 g, preferably from 0.4 g to 1.5 g, of an extract of thyme (Thymus vulgaris) per 100 g of the composition.

Particularly preferably, the non-polar phase (and the composition) contains, per 100 g of the composition, from 0.1 g to 4.0 g, preferably from 1.0 g to 2.5 g, of an extract from the field mint (Mentha arvensis), from 0.1 g to 2.0 g, preferably from 0.2 g to 1.2 g, of an extract of clove tree (Syzygium aromaticum), from 0.1 g to 2.0 g, preferably from 0.2 g to 0.8 g, of an extract of ginger (Zingiber officinale), and from 0.1 g to 2.0 g, preferably from 0.4 g to 1.5 g, of an extract of thyme (Thymus vulgaris).

The advantages of these extracts are described above. In these concentrations, the extracts are particularly useful.

Preferably, the composition contains from 0.05 g to 2.0 g, preferably from 0.1 g to 0.8 g, of an extract from grapes (Vitis vinifera), preferably from grape seeds, per 100 g of the composition. Such an extract contains tannins. Tannins are polyphenols that may have antioxidant, astringent, and enzyme inhibitory properties. Tannins can also impart a special mouthfeel. Certain tannins have been reported to be able to prevent the docking of a virus to its host cell, potentially reducing the risk of infection by pathogens resulting in local or general disruption of the organism. It is possible, therefore, that the composition according to the disclosure can support the defense against pathogens. Because of the formulation of the composition, the composition adheres to the site of application for a relatively long time and can act. When formulated as a spray, the composition can be applied directly and finely dispersed.

Preferably, the non-polar phase (and the composition) contains from 0.5 g to 5.0 g, preferably from 1 g to 3.0 g, of triacylglycerols per 100 g of the composition in addition to any triacylglycerols optionally contained in the extract(s). Preferably, these are medium-chain triacylglycerols, e.g. from coconut oil. Triacylglycerols can serve as carriers for lipophilic active ingredients and essential oils. Their use can stabilize the formulation. Furthermore, the spray behavior of the composition can be influenced in a positive way. Furthermore, the triacylglycerols form an oil film that adheres to the sprayed body part (e.g. throat) over a longer period of time, through which lipophilic compounds can diffuse and act.

Preferably, the composition contains at least one vitamin selected from vitamin B2, vitamin B7 (biotin), vitamin B12, vitamin D3 and derivatives thereof, vitamin A and derivatives thereof, and vitamin E and derivatives thereof per 100 g of the composition. More preferably, the composition contains vitamin B2, vitamin B7 (biotin), vitamin B12, vitamin D3 or a derivative thereof, vitamin A or a derivative thereof, and vitamin E or a derivative thereof. In an even more preferred embodiment, the composition contains, per 100 g of the composition, from 0.1 g to 0.3 g of vitamin B2, from 0.004 g to 0.01 g of vitamin B7 (biotin), from 0.0002 g to 0.0006 g of vitamin B12, from 10000 IU to 50000 IU of vitamin D3, from 0.05 g to 0.2 g of retinyl palmitate, and from 1 g to 3 g of vitamin E acetate. Vitamin A, vitamin D3 and vitamin B12 may contribute to normal immune system function. Vitamin B2, vitamin B7 (biotin) and vitamin A may contribute to the maintenance of normal mucous membranes. Vitamin E may help protect cells from oxidative stress. These vitamins promote general health, especially the health of the cells of the mouth and throat. These vitamins are partially water soluble and partially fat soluble. As a result of the special formulation, these vitamins can be applied simultaneously directly to the mucous membranes of the throat.

In a particularly preferred embodiment, the composition consists of a polar phase and a non-polar phase,

wherein the polar phase contains at least 20% by weight of water based on the total weight of the polar phase, wherein the polar phase contains, based on the total weight of the polar phase, from 40% by weight to 78% by weight, more preferably from 45% by weight to 75% by weight, even more preferably from 50% by weight to 70% by weight, especially preferably from 53% by weight to 67% by weight, most preferably from 56% by weight to 64% by weight, of glycerol,

wherein the composition contains, in addition to water and glycerol, per 100 g of the composition:

from 0.5 g to 4 g, more preferably from 1 g to 3.5 g, even more preferably from 1.5 g to 3.0 g or from 2.0 g to 3.0 g, lecithin (0.2 g to 4 g, more preferably 0.3 g to 3.5 g, even more preferably 0.5 g to 3.0 g or 0.6 g to 3.0 g, phosphatidylcholine or 1 g to 3 g, preferably 1 g to 2 g, phosphatidylcholine),
0.1 g to 4.0 g, preferably 1.0 g to 2.5 g, of an extract of field mint (Mentha arvensis),
0.1 g to 2.0 g, preferably 0.2 g to 1.2 g, of an extract from the clove tree (Syzygium aromaticum),
0.1 g to 2.0 g, preferably 0.2 g to 0.8 g, of an extract of ginger (Zingiber officinale),
0.1 g to 2 g, preferably 0.4 g to 1.5 g, of an extract of thyme (Thymus vulgaris),
0.5 g to 5.0 g, preferably 1.0 g to 3.0 g, triacylglycerols, preferably medium-chain triacylglycerols,
0.01 g to 2.0 g polysaccharides, preferably β-glucans, particularly preferably (1,3)-(1,6)-beta-D-glucans,
0.05 g to 2.0 g, preferably 0.1 g to 0.8 g, of an extract from grapes (Vitis vinifera), preferably from grape seeds,
vitamin B2, preferably 0.1 g to 0.3 g vitamin B2,
vitamin B7 (biotin), preferably 0.004 g to 0.01 g vitamin B7 (biotin),
vitamin B12, preferably 0.0002 g to 0.0006 g of vitamin B12,
vitamin D3, preferably 10000 IU to 50000 IU vitamin D3,
retinyl palmitate, preferably 0.05 g to 0.2 g retinyl palmitate, and
vitamin E acetate, preferably 1 g to 3 g vitamin E acetate.

In a particularly preferred embodiment, the composition consists of these ingredients, with water and glycerol forming the difference ad 100 g of the composition.

In a preferred embodiment, the base of a composition according to the disclosure is formed from a composition containing, per 100 g of the composition:
25 g to 35 g of water,
0.5 g to 4 g lecithin,
1 g to 2.5 g of mint oil (Mentha arvensis),
0.2 g to 1.2 g clove oil (Syzygium aromaticum),
0.2 g to 0.8 g ginger rhizome CO₂-destraction extract (Zingiber officinale),
0.4 g to 1.5 g thyme oil (Thymus vulgaris) and at least 50 g of glycerol. The composition may be modified as disclosed herein.

In a very particularly preferred embodiment, the composition consists, per 100 g of the composition, of:
33 g water,
2 g lecithin (1.5 g phosphatidylcholine),
1.6 g mint oil (Mentha arvensis),
0.4 g clove oil (Syzygium aromaticum),
0.4 g ginger rhizome CO₂-destraction extract (Zingiber officinale),
0.8 g thyme oil (Thymus vulgaris),
2 g medium-chain triacylglycerols,
0.05 g (1,3)-(1,6)-beta-D-glucans,
0.16 g of an extract from grape seeds (Vitis vinifera),
0.2 g vitamin B2,
0.007 g vitamin B7 (biotin),
0.0004 g vitamin B12,
10000 IU to 50000 IU vitamin D3,
0.13 g retinyl palmitate,
vitamin E acetate, preferably 2 g vitamin E acetate, and
glycerol ad 100 g.

In a preferred embodiment, the composition contains no monosaccharide and no disaccharide. In a preferred embodiment, the composition contains a total of at most 1 g of monosaccharides and disaccharides per 100 g of the composition. A monosaccharide in the sense of this specification is, for example, glucose or fructose. A disaccharide within the meaning of this document is, for example, sucrose.

In a preferred embodiment, the composition does not contain a monohydric alcohol. In a preferred embodiment, the composition contains a total of at most 1 g of monohydric alcohols per 100 g of the composition. Monohydric alcohols as defined herein include, for example, ethanol and propanol.

In a preferred embodiment, the composition does not contain a dihydric alcohol. In a preferred embodiment, the composition contains a total of at most 1 g of dihydric alcohols per 100 g of the composition. A divalent alcohol as defined herein is, for example, propylene glycol.

In a preferred embodiment, the composition contains no synthetic surfactant. The term “no” should be understood to mean that the concentration of the compound is so low that no appreciable effect is expected. In other words, the concentration is outside the range at which the compound is normally used. For the purposes of the disclosure, synthetic surfactant means ethoxylated surfactants, macrogol and derivatives thereof (e.g. macrogol glycerol fatty acid esters such as macrogol glycerol ricinoleate, macrogol fatty acid esters such as, e.g., macrogol oleate, macrogol fatty alcohol ethers such as polyoxyethylene cetyl ether), propylene glycol and derivatives thereof, polyoxypropylene-polyoxyethylene block polymers, cellulose and derivatives thereof (e.g. hydroxypropyl methyl cellulose), benzalkonium, benzethonium, cetylalkonium, cetylpyridinium, cetyltrimethylammonium, dequalinium, sodium dodecyl sulfate, sodium cetyl sulfate, sodium stearyl sulfate, ethylene glycol monostearate, partial fatty acid esters of sorbitan (e.g. sorbitan monolaurate, sorbitan monopalmitate, sorbitan monostearate, sorbitan tristearate, sorbitan monooleate, sorbitan sesquioleate, sorbitan trioleate, polysorbates (polyoxyethylene sorbitan fatty acid esters), fatty acid esters of sucrose, fatty acid esters of polyglycerol, and D-α-tocopheryl-1000-succinate.

In a preferred embodiment, the composition contains no artificial preservative. The term “no” should be understood to mean that the concentration of the compound is so low that no appreciable effect is expected. In other words, the concentration is outside the range at which the compound is normally used. For the purposes of the disclosure, artificial preservatives include 4-hydroxybenzoic acid esters (e.g. 4-hydroxybenzoic acid methyl ester and 4-hydroxybenzoic acid propyl ester), chlorocresol, chlorobutanol, sorbic acid (E 200), potassium sorbate (E 202), calcium sorbate (E 203), sodium edetate, glycerol triacetate, benzalkonium, benzethonium, cetylalkonium, cetylpyridinium, cetyltrimethylammonium, cetrimonium, chlorhexidine, dequalinium, organic mercury compounds.

In a preferred embodiment, the composition contains no artificial sweetener. The term “no” should be understood to mean that the concentration of the compound is so low that no appreciable effect is expected. In other words, the concentration is outside the range in which the compound is normally used. Artificial sweeteners within the meaning of the disclosure include acesulfame (E 950), advantame (E 969), aspartame (E 951), aspartame-acesulfame salt (E 962), cyclamate (E 952), neohesperidin (E 959), neotame (E 961), saccharin (E 954) and sucralose (E 955). Steviol glycosides, e.g. stevioside (E 960), are not considered artificial sweeteners within the meaning of the disclosure.

In a preferred embodiment, the composition is sugar-free within the meaning of Regulation (EC) No 1924/2006 of the European Parliament and of the Council of 20 Dec. 2006 on nutrition and health claims made on foods. In other words, in a preferred embodiment, the composition contains no more than 0.5 g of sugar (monosaccharides and disaccharides) per 100 g of the composition.

In a particular embodiment, the composition does not contain any ingredients of animal origin. In other words, the composition is suitable for a vegetarian and/or vegan lifestyle.

From the efforts to solve the object mentioned in the introduction, there further results a spray comprising a spray device and a storage chamber, wherein the storage chamber contains a composition according to the disclosure. The spray device has means for guiding the composition according to the disclosure out of the storage chamber and delivering it to the environment in the form of fine droplets or an atomizing jet. Suitable sprays comprising a spray device and a storage chamber are known to the person skilled in the art and the user, for example, from mouth, throat and pharynx sprays from the pharmacy or drugstore. By actuating, preferably manually depressing, a trigger on the spray device (atomizer), the user can deliver a spray of a liquid with a typically aqueous or ethanolic base from the storage chamber in the form of fine droplets or an atomizing jet to the environment. A spray according to the disclosure comprises a composition according to the disclosure in such a commercially available device comprising a sprayer and a storage chamber.

The efforts to solve the object mentioned in the introduction result in the use of a spray device for distributing a composition according to the disclosure.

Preferably, the distribution is a spray.

Preferably, the use according to the disclosure is a non-medical use.

From the efforts to solve the object stated in the introduction, there further results a non-medical method for conditioning a portion of the human body to achieve at least one effect selected from i) assisting in maintaining the function of the mucosa, ii) assisting in the function of the immune system, iii) assisting in protecting cells from oxidative stress, iv) improving breath odor (halitosis), v) improving taste, vi) improving mouthfeel, and vii) assisting in defense against pathogens selected from viruses and bacteria, wherein the method comprises applying to the portion of the human body a composition according to the disclosure.

Preferably, the application is a spraying, preferably by means of a spray device (of a spray).

The section of the human body is preferably selected from the oral cavity and pharynx.

The disclosure is further illustrated below by means of an example.

An exemplary composition according to the disclosure contains per 100 g of the composition:
33 g water,
2 g lecithin (1.5 g phosphatidylcholine),
1.6 g mint oil (Mentha arvensis),
0.4 g clove oil (Syzygium aromaticum),
0.4 g ginger rhizome CO₂-destraction extract (Zingiber officinale),
0.8 g thyme oil (Thymus vulgaris),
2 g medium-chain triacylglycerols,
0.05 g (1,3)-(1,6)-beta-D-glucans,
0.16 g of an extract from grape seeds (Vitis vinifera),
0.2 g vitamin B2,
0.007 g vitamin B7 (biotin),
0.0004 g vitamin B12,
10000 IU to 50000 IU vitamin D3,
0.13 g retinyl palmitate,
2 g vitamin E acetate and
Glycerol ad 100 g.

The composition is liquid or a liquid at room temperature (25° C.). All ingredients occur naturally. The composition does not contain significant amounts of monosaccharide, disaccharide, mono or dihydric alcohol, synthetic surfactant, artificial preservative or artificial sweetener. Ingredients of animal origin are not required. The composition has a low risk of intolerances such as allergies. The composition exhibits good taste and odor, is non-cariogenic and does not cause blood sugar spikes. The composition is sugar-free within the meaning of Regulation (EC) No 1924/2006 of the European Parliament and of the Council of 20 Dec. 2006 on nutrition and health claims made on foods. In other words, the composition contains no more than 0.5 g of sugar (monosaccharides and disaccharides) per 100 g of the composition.

At the same time, the composition is microbiologically and physically stable. When used in a spray, the composition can be sprayed into the human throat via a common spray device and is suitable to be swallowed without hesitation. The special base of glycerol, water, medium-chain triacylglycerols and lecithin in certain proportions allows adherence to the throat and long interaction and exchange between the composition and sprayed body part.

The vitamins promote general health, especially the health of the cells of the oral pharynx. Thanks to the special formulation, the water-soluble and fat-soluble vitamins can be applied directly to the mucous membranes with one spray, supporting mucous membranes and the immune system.

By using the extract(s), the proportion of preservative glycerol in the composition can be reduced, thus increasing the proportion of water. Among other things, this makes the composition sprayable. In addition to the preservative effect, ingredients of the extract(s) exhibit antioxidant and possibly antiviral and/or antibacterial effects. In addition, the composition improves taste, breath odor (halitosis), and mouthfeel. The person skilled in the art will recognize that the individual ingredients of the composition interact in a particular way to form a product according to the disclosure with all its beneficial properties.

1. Composition consisting of a polar phase and a non-polar phase, wherein
the polar phase contains at least 40% by weight of glycerol and at least 20% by weight of water, based on the total weight of the polar phase,
the composition contains from 0.5 g to 4 g of lecithin per 100 g of the composition, and
the non-polar phase contains at least one extract selected from an extract of mint (Mentha arvensis or Mentha x piperita), an extract of clove tree (Syzygium aromaticum), an extract of ginger (Zingiber officinale) and an extract of thyme (Thymus vulgaris).

2. Composition according to claim 1, wherein the non-polar phase contains an extract of mint (Mentha arvensis or Mentha x piperita), an extract of clove tree (Syzygium aromaticum), an extract of ginger (Zingiber officinale) and an extract of thyme (Thymus vulgaris).

3. Composition according to claim 1, wherein the non-polar phase contains an extract of field mint (Mentha arvensis), an extract of clove tree (Syzygium aromaticum), an extract of ginger (Zingiber officinale) and an extract of thyme (Thymus vulgaris).

4. Composition according to claim 1, wherein
the polar phase contains, based on the total weight of the polar phase, 40% by weight to 78% by weight of glycerol, and
the non-polar phase contains from 0.4 g to 10.0 g of the extract or extracts in total per 100 g of the composition.

5. Composition according to claim 1, wherein the composition contains from 0.01 g to 2.0 g polysaccharides per 100 g of the composition.

6. Composition according to claim 1, wherein the non-polar phase contains from 0.1 g to 4.0 g of an extract from the field mint (Mentha arvensis) per 100 g of the composition.

7. Composition according to claim 1, wherein the non-polar phase contains from 0.1 g to 2.0 g of an extract from the clove tree (Syzygium aromaticum) per 100 g of the composition.

8. Composition according to claim 1, wherein the non-polar phase contains from 0.1 g to 2.0 g of an extract from ginger (Zingiber officinale) per 100 g of the composition.

9. Composition according to claim 1, wherein the non-polar phase contains from 0.1 g to 2.0 g of an extract of thyme (Thymus vulgaris) per 100 g of the composition.

10. Composition according to claim 1, wherein the composition contains from 0.05 g to 2.0 g of an extract from grapes (Vitis vinifera) per 100 g of the composition.

11. Composition according to claim 1, wherein the non-polar phase contains from 0.5 g to 5.0 g of triacylglycerols per 100 g of the composition in addition to triacylglycerols contained in the extract(s).

12. Composition according to claim 1, wherein the composition contains at least one vitamin selected from vitamin B2, vitamin B7 (biotin), vitamin B12, vitamin D3 and derivatives thereof, vitamin A and derivatives thereof, and vitamin E and derivatives thereof.

13. Spray, comprising a spray device and a storage chamber, wherein the storage chamber contains a composition according to claim 1.

14. Use of a spray device for distributing a composition according to claim 1.

15. Non-medical method of conditioning a portion of the human body to achieve at least one effect selected from i) assisting in maintaining the function of the mucosa, ii) assisting in the function of the immune system, iii) assisting in protecting cells from oxidative stress, iv) improving breath odor, v) improving taste, vi) improving mouthfeel, and vii) assisting in defense against pathogens selected from viruses and bacteria, wherein the method comprises applying to the portion of the human body a composition according to claim 1.

16. Composition according to claim 4, wherein
the polar phase contains, based on the total weight of the polar phase, 56% by weight to 64% by weight, of glycerol.

17. Composition according to claim 4, wherein the non-polar phase contains from 1.5 g to 7.0 g of the extract or extracts in total per 100 g of the composition.

18. Composition according to claim 5, wherein the polysaccharides are (1,3)-(1,6)-beta-D-glucans.

19. Composition according to claim 8, wherein the extract is a CO₂-distraction extract.

20. Composition according to claim 10, the extract is frm grape seeds.
